(19) United States
(12) Patent Application Publication
(10) Pub. No.: US 2022/0064854 A1
(43) Pub. Date: Mar. 3, 2022

(54) UV LIGHT SANITIZATION OF CARPETING

(71) Applicant: COLUMBIA INSURANCE COMPANY, Omaha, NE (US)

(72) Inventors: THOMAS ODUM, DALTON, GA (US); STEVEN R. DUNKLE, DALTON, GA (US)

(21) Appl. No.: 17/459,216

(22) Filed: Aug. 27, 2021

Related U.S. Application Data
(60) Provisional application No. 63/071,648, filed on Aug. 28, 2020.

(51) Int. Cl. D06N 7/00; D02G 3/44; D02G 3/04; A61L 2/10

(52) U.S. Cl. CPC D06N 7/0065; D06N 7/0068; D02G 3/445; A61L 2202/26; A61L 2/10; D06N 2209/0861; D06N 2211/066; D02G 3/04

(57) ABSTRACT

A healthier carpet employing UV light-transmitting threads, which facilitate UV light transmittance down from the surface of the carpet, through the interior of the carpet, and into the primary backing of the carpet. The UV light can destroy bacteria, molds, viruses, yeast, and other microorganisms, which are present in the carpet. An external UV light source, with UV wavelengths from 100-400 nm, can be passed over the carpet for effective sanitization.

### CROSS-REFERENCE TO RELATED APPLICATION

This application claims the benefit of priority to U.S. Provisional Patent Application No. 63/071,648, filed on Aug. 28, 2020, the entirety of which is incorporated herein by reference.

### BACKGROUND

Carpeting is a beneficial addition to many homes and businesses. However, over time, unwanted infectants from the environment may settle upon the carpeting. Current methods for cleaning and sanitizing carpeting are ineffective at the neutralization of these unwanted infectants on a daily basis.

For example, one method of cleaning carpeting is vacuuming, which involves lifting particles from the carpet by suction. While this technique may be useful in removing loose particles in the carpeting, it does not sanitize the carpeting to destroy environmental infectants, such as microbes, viruses and fungi

Another method of carpet cleaning is steam cleaning, which involves spraying warm water and surfactants onto the carpeting followed by suctioning. This process, if done incorrectly, can leave the carpeting damp. In addition, this carpet cleaning method does not eliminate microbes, viruses, or fungi on a continuous or daily basis.

Thus, there exists a need for a safe and effective method to sanitize carpeting, which the present disclosure provides.

### BRIEF SUMMARY

The present application is directed to carpeting containing yarns with UV light-transmitting threads, which facilitate transmittance of UV light down the length of the thread and into the backing of the carpet, for eco-friendly, effective sanitization of the top, middle, and lower portions of the carpet. A device with UV light output is placed over the carpet to complete the sanitization process.

One embodiment includes a carpet made from (a) at least one UV light-transmitting thread; (b) a carpet fiber; and (c) a primary backing to which the UV light-transmitting thread and carpet fiber are attached. The carpet can have a secondary backing and an adhesive adhering the primary backing to the secondary backing.

In one embodiment, the UV light-transmitting thread is an aliphatic polymer. In another embodiment, the UV light-transmitting thread is a polymer, such as cyclic olefin copolymers, polymethylpentene polymer, fluoropolymers or silicone based polymers or a mixture thereof. It is noted that the above-mentioned examples of the UV-light transmitting thread are non-limiting, and the UV-light transmitting thread can include any appropriate polymers that can both transmit UV light (or UV-C light) and is not degraded by said transmission of the UV light (or UV-C light) over time. In some example embodiments, the UV-light transmitting thread can include UV-C transparent polymers. The UV-light transmitting thread may include stabilizers that prevents or mitigates the degradation of the thread from exposure to or transmission of UV and/or UV-C light therethrough. The cross-sectional geometry of the UV light-transmitting thread is circular, triangular, rectangular, multi-lobal, window pane, hollow, bi-axial fibers or a mixture thereof. The UV light-transmitting thread is about 4-25 denier.

In certain embodiments, the carpet fibers are natural fibers, polymeric fibers, or a mixture thereof. In an embodiment, the carpet fiber is acrylic fibers, nylon fibers, polyester fibers, or a mixture thereof. The carpet fiber is about 4-25 denier. In some embodiments, the carpet fibers and/or primary backing can contain a UV-protection agent. UV-protection agents can include about 0.5 to about 1.0 wt. % of a hindered phenolic antioxidant, about 0.05 to about 1.5 wt. % of a phosphite compound, about 0.05 to about 1.5 wt. % of a hindered amine light stabilizer, or about 0.05 to about 5.0 wt. % of a benzotriazole UV absorber. One or more UV-protection agents can be added during the fiber extrusion process or afterwards.

In some embodiments, the UV light-transmitting thread transmits light of about 100-400 nm, which covers the entire range of UV light. In certain embodiments, the UV light-transmitting thread transmits UV light of about 200-280 nm, which corresponds to a range of UV-C light capable of destroying or disrupting bacteria, viruses, dust-mites, and other microorganisms to an extent that renders them ineffective.

The carpet has yarn tufted into the primary backing wherein the yarn is composed of at least one strand that comprises a bundle of carpet fibers, UV light-transmitting threads, or a mixture thereof. In another embodiment, the yarn is made of (a) at least one strand comprising a mixture of carpet fibers and UV light-transmitting threads; (b) at least one strand comprising carpet fibers, (c) at least one strand comprising UV-transmitting threads; or (d) a mixture of (a), (b), and (c). This means that some tufts of yarn can contain no UV light-transmitting threads, some contain solely UV light-transmitting threads, and some contain a mixture of both. The tufts containing the UV-transmitting threads can be placed uniformly or randomly in the primary carpet backing. A carpet contemplated herein will have some UV light-transmitting threads as a bundle of a mixture of carpet fibers and UV light-transmitting threads. Alternatively, the yarn is made of separate strands comprising a bundle of carpet fibers and separate strands comprising a bundle of UV light-transmitting threads, which strands are intertwined. In certain embodiments, each bundle of fibers with UV light-transmitting threads contains about 2-20 UV light-transmitting threads.

The primary backing of the carpet is polyester or polypropylene. The secondary backing, which is optional, can be the same or different material than the primary backing.

Also included herein is a method of making a carpet comprising UV light-transmitting threads, which includes (a) adding UV light-transmitting threads during a fiber spinning process for carpet fibers or after drawing of the carpet fibers; (b) intermingling the carpet fibers and the UV light-transmitting threads to produce yarn; and (c) tufting the yarn into a primary backing wherein the tufts can be continuous loop or cut pile.

In order to sanitize the carpeting, a high output UV light (e.g., short wave UV light (UV-C)) is passed over the carpet for a period of time to effectively illuminate the UV light-transmitting threads, which transmit the UV light from the surface of the carpet all the way down to the primary backing and/or the secondary backing. The UV light will damage and/or kill microorganisms, molds, fungi, viruses, and dust-mites residing within the carpeting without the need to apply surfactants.

The carpeting described herein has several benefits and advantages. One benefit is that the carpeting contains UV light-transmitting threads, which can transmit UV light to effectively kill bacteria and other microorganisms. Another benefit is that the UV light-transmitting threads facilitate the penetration of UV light deep down into the carpet where the great majority of microorganisms exist to thoroughly sanitize the carpeting at all depths and not just the surface. Yet another benefit is that dirt and debris present in the carpeting cannot interfere with the penetration of UV light because the fibers have an integrated light pathway within a substantially impermeable, polymer material. Still another benefit is that there is no need for large amounts of water and surfactants because the carpet can be sanitized in an eco-friendly manner, simply by the application of UV light. Another benefit is that the UV light does not add anything to the carpet, such as undesirable odor, color, or residual matter.

### BRIEF DESCRIPTION OF THE DRAWINGS

FIG. 1 is a view of a cross-section of carpeting containing UV light-transmitting threads.

FIG. 2 is a view of a tuft made of both carpet fibers and UV light-transmitting threads.

FIG. 3 is a view of the yarn made from strands of both carpet fibers and UV light-transmitting threads in a carpet.

FIG. 4 is a view of UV light-transmitting threads tufted separately into the primary backing as well as tufts of carpet fibers and carpet fibers with UV light-transmitting threads.

### DETAILED DESCRIPTION OF EXEMPLARY EMBODIMENTS

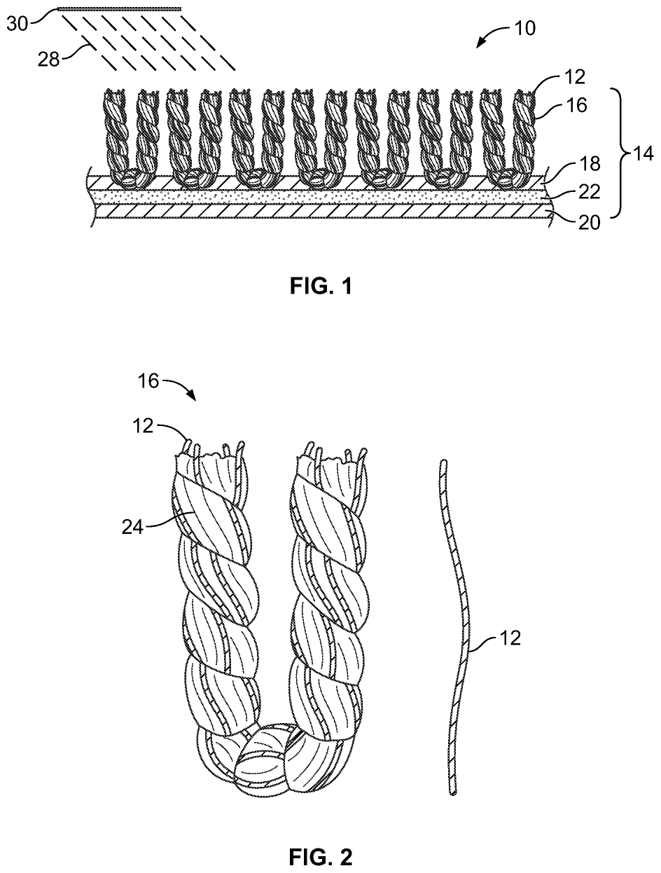

FIG. 1 shows a particular embodiment of a carpeting assembly 10 containing UV light-transmitting threads 12 being illuminated with UV light 28 from a UV lamp 30. The carpet 14 has yarn 16 tufted into a primary backing 18. A secondary backing 20, which is optional, can be underneath the primary backing 18. An adhesive layer 22 adheres the primary backing 18 to the secondary backing 20 and keeps the yarn 16 stationary.

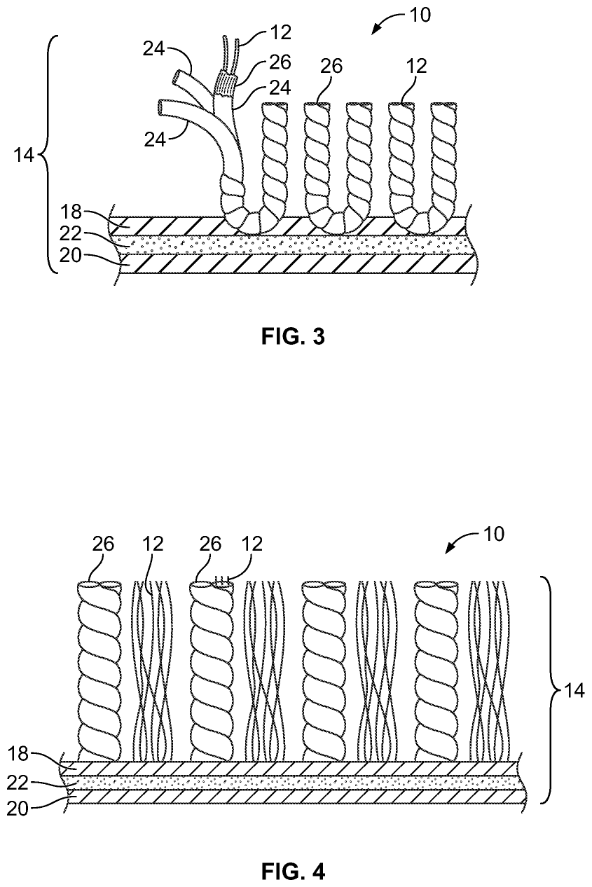

The yarn 16 is made of at least one strand 24, commonly at least two to six strands 24, that are twisted together, as shown in FIG. 2. As shown in FIG. 3, the strands 24 are made from a bundle of carpet fibers 26, UV light-transmitting threads 12, or a mixture thereof. The yarn 16 can be made from a mixture of strands 24, such as (a) at least one strand 24 of carpet fibers 26, (b) at least one strand 24 of UV light-transmitting threads 12, (c) at least one strand 24 of a mixture of carpet fibers 26 and UV light-transmitting threads 12 or (d) a mixture of (a), (b), and (c), as also shown in FIG. 3.

In one embodiment, some of the strands 24 contain only carpet fibers 26 and do not contain a UV light-transmitting thread 12, other strands 24 contain only light transmitting threads 12, yet other strands 24 contain both carpet fiber 26 and UV light-transmitting threads 12. A strand 24 can have about 2-20 UV light-transmitting threads 12.

In another embodiment, the UV light-transmitting threads 12 can be tufted separately into the primary backing 18 without being in a yarn 16 with the other tufts being all carpet fibers 26 or a mixture of carpet fibers 26 and UV light-transmitting threads 12, as shown in FIG. 4. In each embodiment, the carpeting assembly 10 contains at least one UV light-transmitting thread 12 either tufted as a mixed yarn 16 or tufted separately as a bundle, group, or individual UV light-transmitting threads 12.

In general, the yarn 16 can be almost any length, including about 0.5-6 inches. The carpet fibers 26 and the UV light-transmitting threads 12 can be the same length or different lengths. The top surface of the UV light-transmitting threads 12 can be flush with the top of the carpet fibers 26. The top surface of the UV light-transmitting threads 12 can be lower than or higher than the top of the carpet fibers 26, or some sections of the carpeting can have the top surface of the UV light-transmitting threads 12 the same as, lower than, or higher than the top of the carpet fibers. There also can be a mixture of different carpet fibers 26 and different UV light-transmitting threads 12 of various sizes, lengths, deniers, and compositions. The UV light-transmitting threads 12 can be comprised of an aliphatic polymer. In another embodiment, the UV light-transmitting threads 12 can be comprised of a polymer, such as cyclic olefin copolymers, polymethylpentene, fluoropolymers or silicone based polymers, or a mixture thereof. The cross-sectional geometry of the UV light-transmitting thread 12 can be circular, triangular, rectangular, multi-lobal, or a mixture thereof. The UV light-transmitting thread 12 is about 4-25 denier.

Generally, the carpet fibers 26 start out as having a natural polymer color with an added delusterant, such as TiO₂ when tufted into a backing. The UV light-transmitting threads 12 have a transparent appearance. The carpet fibers 26 are then dyed a specific color in the carpet finishing process. Typically, the entire tufted carpet is dyed using a continuous dye range where the entire width of the tufted carpet from 12 ft. to 16 ft. wide is dyed in a continuous method as it is conveyed through the dyeing section. The dye is flooded onto the carpet and dyes are exhausted into the fibers. The carpet is then washed and dried for subsequent processes. In one embodiment, the carpet fiber is pre-dyed where the pigment is added in the carpet fiber extrusion process to create a finished fiber color. In another instance, the carpet fibers 26 are dyed using a skein dye method for creating color of the fibers. This involves creating skeins of fiber, then vat dying the skeins. In another embodiment, the carpet fibers 26 are dyed using a space dye method. The yarn can be dyed with many colors along the axis of the fibers to create a unique look. This can be accomplished by knitting the fibers into a form, then applying a dye, then deknitting. Other unique looks can be accomplished by a machine method of jetting or creating pre-programmed and random dye spots.

The carpet fiber 26 can be natural fibers, polymeric fibers, or a mixture thereof. Natural fibers include cotton, wool, hemp, silk, cellulose, or a mixture thereof. Polymeric fibers include polyester, acrylic, nylon, polypropylene, polystyrene, or a mixture thereof. Similar to the UV light-transmitting thread 12, the carpet fiber 26 is about 4-25 denier. The denier of the UV light-transmitting threads 12 and the carpet fiber can be the same or different.

The carpet fibers 26 can optionally contain a UV-protection agent. UV-protection agents can include about 0.5 to about 1.0 wt. % of a hindered phenolic antioxidant, about 0.05 to about 1.5 wt. % of a phosphite compound, about 0.05 to about 1.5 wt. % of a hindered amine light stabilizer, or about 0.05 to about 5.0 wt. % of a benzotriazole UV absorber. One or more UV-protection agents can be added during the fiber extrusion process or afterwards.

In one aspect, the UV light-transmitting thread 12 transmits light of about 100-400 nm. In another aspect, the UV light-transmitting thread 12 transmits light of about 100-280 nm. The UV light kills microorganisms, such as bacteria, viruses, molds, and yeast and can sanitize the carpeting with minimal labor. In particular, light of 200-280 nm is known as UV-C light, kills bacteria and other microorganisms.

To sanitize the carpet 14, an external UV lamp 30 is placed over the carpet 14 having the UV light-transmitting threads 12 for a period of time to irradiate the carpet 14. The UV lamp 30 can be part of a device on wheels that can be easily rolled over the carpet 14. The UV lamp 30 can be hand-held or part of a robotic device or tool. The treatment time will vary depending on the strength of the UV lamp 30 and the distance from the carpet 14 surface. Also, the time will depend on the total amount of microorganisms present in the carpet 14 and/or the number of UV light-transmitting threads 12 that are in the carpet 14. The UV dosage can be expressed as a product of the UV intensity and the exposure or residence time. One common dosage unit is microwatt-seconds per centimeter squared. The effectiveness of the UV light 28 is based on the microwatt exposure of the microbe to the light. Carpets 14 having less UV light-transmitting threads 12 will require more time than those with more UV light-transmitting threads 12.

The UV light 28 damages/disrupts the DNA and/or kills microorganisms, and sanitizes the rug when the UV light 28 penetrates the cells of the microorganism causing death. The term UV light 28 as used herein may include light falling in the UV wavelength range of 100-400 nm. For example, the UV light 28 can include, UV-A, UV-B, and/or UV-C. In some example embodiments, UV-C light may be used to damages/disrupts the DNA and/or kills microorganisms. Sanitization refers to a reduction of the total number of microorganisms and is not a sterilization, which is the elimination of all microorganisms. In general, UV disinfection can be measured as a function of UV dose. A UV dosimeter can be used to monitor sterilization. Many common microorganisms can be killed using a UV dosage of less than 15,000 microwatt-seconds per centimeter squared. As such, in certain example embodiments, a UV light having a dosage of less than or equal to 15,000 microwatt-second per centimeter squared may be used for sanitization. In some example embodiments, UV light having a dosage of less than or equal to 7,000 microwatt-second per centimeter squared may be used for sanitization. In yet other example embodiments, UV light having a dosage that is higher than 15000 microwatt-second per centimeter squared may be used to the extent that it does not exceed a maximum acceptable dosage for safe usage of human beings and/or it does not negatively impact the structure of the different fibers used within the carpet.

In one example, the average carpet face fiber is made from either polyester, nylon, or polypropylene. Typically, there would be anywhere from 1 to 10 UV light-transmitting filaments per bundle of carpet fiber. In some instances, two bundles of fiber would be twisted together and heatset prior to tufting into the primary backing. Adhesive can be applied and a secondary backing would be added to the construction. After the carpet or carpet tile is in service, a UVC lamp, typically a mercury vapor lamp which emits more than 90% of its radiation at 253.7 nm, is used to sanitize the carpet or carpet tile. A pulsed Xenon lamp can be used also. Generally, UV light exposure time ranges from 5 to 15 minutes to sanitize an average room of carpeting.

The UV light-transmitting threads 12 provide an unimpeded pathway for the light to travel from the surface of the carpet 14 to the primary backing 18 because the light path is part of a nonporous polymer thread. In some circumstances, the primary backing 18 is made of UV light-transmitting material also, as is the adhesive 22. The primary backing 18 can be polyester or polypropylene. In some instances, hotmelt adhesives are used to adhere fibers and backing layers together. These adhesives can have light transmissive properties. Also, specialized backing materials with low fillers and pigments can be used to facilitate light transmission too. The backing materials can be made from polyethylene terephthalate (PET), polypropylene (PP), and other polymers that are visually transparent.

The primary backing 18, the adhesive 22, and the carpet fibers 26 can contain an additive, such as a UV-protection agent, a dye, an antimicrobial agent, or a mixture thereof.

Also included herein is a method of making a carpet comprising UV light-transmitting threads, which includes: adding the UV light-transmitting threads during a fiber spinning process for carpet fibers; adding the UV light-transmitting threads after drawing of the carpet fibers; adding the UV light-transmitting threads in the extrusion process and having the aligned threads become part of the overall filament count of the yarn; adding the UV light-transmitting threads on a carrier tube or cone into the creel yarn during the direct cabling process; intermingling the carpet fibers and the UV light-transmitting threads to produce yarn; and tufting the yarn into a primary backing wherein the tufts can be continuous loop or cut pile.

There are additional methods for introducing UV light-transmitting threads into a yarn bundle. In another example, staple fibers are cut to specific lengths from about 1″ to 6″ then processed using a carding and spinning method that creates longer length fibers from short length fibers. The carpet fibers, UV light-transmitting threads, and/or mixtures thereof created by any of these methods are then tufted into a primary backing cloth having a woven pattern of fibers or a spun bond matrix of fibers. In the tufting process, the carpet fibers, UV light-transmitting threads, and/or mixtures thereof are pushed through the primary backing material to create a loop of yarn on the opposite side of the primary backing. The end of this newly created loop is held in place by a device as the needle which pushes the yarn through the primary backing is withdrawn. This leaves a loop of yarn on the opposite side of the fabric. In some instances, the loop that is created on the opposite side of the fabric is cut after the needle is withdrawn. This creates a cut pile tuft on the opposite side. The sequence continues as the fabric is advanced slightly to a new position to create another tuft. This repeated action later becomes the face of the carpet. This now tufted fabric will go for further processing to add dyes to the fiber tufts, then the tufts are held in place in the primary backing by an adhesive. In some instances, this adhesive can be marginally transparent to UV light. Additional fabrics are added to the back of the tufted carpet to provide strength for new formed fabric. The UV transmitting fibers are retained in the tufts by being twisted or parallel with other fibers and then retained in the fabric by using adhesive.

In another aspect, a method of making a carpet having UV light-transmitting thread includes (a) adding UV light-transmitting threads during a fiber spinning process for carpet fibers or after drawing of the carpet fibers; (b) intermingling the carpet fibers and the UV light-transmitting threads to produce yarn; and (c) tufting the yarn into a primary backing wherein the tufts can be continuous loop or cut pile. Other methods for introducing UV transmissive fibers into the yarn bundle can be done in the direct cabling process. A wound package holding the UV light transmissive fiber(s) can be arranged onto the creel of a BCF cabling machine and fed into the yarn path with the creel yarn. It is then direct cabled with the yarn in the bucket section of the machine. In another example the UV light transmitting fiber can be chopped into staple fiber lengths of 1-8″ long and blended with other carpet fiber polymers of similar lengths. The UV light transmissive fiber will be distributed into the fiber blend and processed through a semi worsted spinning process, commonly referred to as spun staple yarn processing.

Alternative embodiments of the subject matter of this application will become apparent to one of ordinary skill in the art to which the present invention pertains, without departing from its spirit and scope. It is to be understood that no limitation with respect to specific embodiments shown here is intended or inferred.

We claim:

1. A carpet comprising:
(a) at least one UV light-transmitting thread;
(b) a carpet fiber; and
(c) a primary backing to which the UV light-transmitting thread and carpet fiber are attached.

2. The carpet of claim 1 further comprising a secondary backing and an adhesive adhering the primary backing to the secondary backing.

3. The carpet of claim 1 wherein the UV light-transmitting thread comprises an aliphatic polymer.

4. The carpet of claim 1 wherein the UV light-transmitting thread comprises a polymer comprising cyclic olefin copolymer, polymethylpentene, fluoropolymer, silicone based polymers, or a mixture thereof.

5. The carpet of claim 1 wherein the cross-sectional geometry of the UV light-transmitting thread comprises circular, triangular, rectangular, multi-lobal, or a mixture thereof.

6. The carpet of claim 1 wherein the UV light-transmitting thread comprises about 4-25 denier.

7. The carpet of claim 1 wherein the carpet fiber comprises natural fibers, polymeric fibers, or a mixture thereof.

8. The carpet of claim 1 wherein the carpet fiber comprises acrylic fibers, nylon fibers, polyester fibers, polypropylene fibers or a mixture thereof.

9. The carpet of claim 1 wherein the carpet fiber comprises about 4-25 denier.

10. The carpet of claim 1 wherein UV light-transmitting thread transmits light of about 100-400 nm.

11. The carpet of claim 1 wherein UV light-transmitting thread transmits light of about 200-280 nm.

12. The carpet of claim 1 comprising yarn tufted into the primary backing wherein the yarn comprises at least one strand comprising carpet fibers, UV light-transmitting threads, or a mixture thereof.

13. The carpet of claim 12 wherein the yarn comprises:
(a) at least one strand comprising a mixture of carpet fibers and UV light-transmitting threads;
(b) at least one strand comprising carpet fibers,
(c) at least one strand comprising UV-transmitting threads; or
(d) a mixture of (a), (b), and (c).

14. The carpet of claim 12 wherein the yarn comprises strands comprising a mixture of carpet fibers and UV light-transmitting threads.

15. The carpet of claim 12 wherein the yarn comprises separate strands comprising carpet fibers and separate strands comprising UV light-transmitting threads, which strands are intertwined.

16. The carpet of claim 12 wherein the strand comprises about 2-20 UV light-transmitting threads.

17. The carpet of claim 1 wherein the primary backing comprises polyester or polypropylene.

18. The carpet of claim 1 wherein the carpet fibers and/or primary backing comprise a UV-protection agent.

19. The carpet of claim 2 wherein the primary backing and/or adhesive are capable of transmitting UV light.

20. A method of making a carpet comprising UV light-transmitting threads comprising:
(a) adding UV light-transmitting threads during a fiber spinning process for carpet fibers or after drawing of the carpet fibers;
(b) intermingling the carpet fibers and the UV light-transmitting threads to produce yarn; and
(c) tufting the yarn into a primary backing.
